(19) United States
(12) Patent Application Publication
(10) Pub. No.: US 2014/0094622 A1
(43) Pub. Date: Apr. 3, 2014

(54) PRODUCTION OF METHYL ACETATE FROM REACTANTS CONTAINING METHYL GROUPS

(71) Applicants: Stephane MARIE-ROSE, Sherbrooke (CA); Esteban CHORNET, Sherbrooke (CA)

(72) Inventors: Stephane MARIE-ROSE, Sherbrooke (CA); Esteban CHORNET, Sherbrooke (CA)

(21) Appl. No.: 13/633,220

(22) Filed: Oct. 2, 2012

(51) Int. Cl. C07C 67/36; C07C 67/37

(52) U.S. Cl. 560/232

(57) ABSTRACT

A method of producing methyl acetate from methanol in which methanol and carbon monoxide are reacted in a first reaction zone to provide a reaction product including methyl acetate, acetic acid, and unreacted methanol. The reaction product then is passed to a second reaction zone, wherein the acetic acid is reacted with the unreacted methanol to provide additional methyl acetate. The method is a more efficient method for producing methyl acetate from methanol in that acetic acid is not separated from the reaction product.

This application claims priority based on provisional Application Ser. No. 61/555,032, filed Nov. 3, 2011, the contents of which are incorporated by reference in their entirety.

This invention relates to the production of methyl acetate from reactants containing methyl groups, such as methanol and dimethyl ether, wherein methanol and/or dimethyl ether is reacted with carbon monoxide to produce methyl acetate and acetic acid, followed by reacting the acetic acid with methanol to produce additional methyl acetate. More particularly, this invention relates to (a) reacting methanol and/or dimethyl ether with carbon monoxide to produce a reaction product including methyl acetate, acetic acid, and unreacted dimethyl ether and/or unreacted methanol, and, (b) without separating the acetic acid from the reaction product of step (a), reacting the acetic acid with methanol to produce additional methyl acetate.

This invention also relates to producing acetates from alcohols by reacting at least one alcohol with carbon monoxide in a first reaction zone to provide a reaction product comprising at least one acetate, at least one acid, and at least one alcohol. The at least one acid then is reacted with the unreacted at least one alcohol in a second reaction zone to provide at least one acetate,

Methyl acetate may be produced by reacting methanol with carbon monoxide (which may be obtained from synthesis gas) in a carbonylation reaction to produce methyl acetate, acetic acid, and water. The main reactions involved in the carbonylation are as follows:

CH₃OH+CO→CH₃COOH

2CH₃OH+CO→CH₃COOCH₃+H₂O

The carbonylation reaction may be carried out in the presence of a suitable catalyst, such as, for example, a rhodium-based catalyst, and a halide promoter, such as methyl iodide or hydrogen iodide.

After the carbonylation reaction is effected, the acetic acid is separated from the reaction product and reacted with methanol to produce additional methyl acetate, according to the following formula:

CH₃COOH+CH₃OH→CH₃COOCH₃+H₂O

The reaction may be conducted in the presence of a suitable catalyst, such as an acid-based catalyst, for example. Examples of reacting methanol with carbon monoxide to produce methyl acetate and acetic acid, and then separating the acetic acid from the reaction product and reacting the acetic acid to make additional methyl acetate are described in published U.S. Patent Application No. US2009/0326080.

In accordance with an aspect of the present invention, there is provided a method of producing methyl acetate from at least one reactant containing at least one methyl group. The method comprises reacting the at least one reactant containing at least one methyl group with carbon monoxide in a first reaction zone to provide a reaction product comprising methyl acetate, acetic acid, and unreacted at least one reactant containing at least one methyl group. The reaction product then is passed to a second reaction zone, whereby the acetic acid is reacted with methanol to provide methyl acetate.

The term “reactant containing at least one methyl group”, as used herein, means any compound which includes, as part of such compound, at least one methyl, or —CH₃, group, other than methyl acetate and acetic acid. Such reactants include, but are not limited to, methanol and dimethyl ether.

In a non-limiting embodiment, the at least one reactant containing at least one methyl group is methanol. Thus, in a non-limiting embodiment, methanol is reacted with carbon monoxide in a first reaction zone to provide a reaction product comprising methyl acetate, acetic acid, and unreacted methanol. The reaction product then is passed to a second reaction zone, whereby the acetic acid is reacted with unreacted methanol to provide methyl acetate.

In a non-limiting embodiment, the methanol is reacted with carbon monoxide in the first reaction zone to provide a reaction product which comprises methyl acetate, acetic acid, unreacted methanol, and water.

In another non-limiting embodiment, the water is removed prior to reacting the acetic acid with the unreacted methanol to provide additional methyl acetate.

In another non-limiting embodiment, the reaction product further comprises unreacted carbon monoxide.

In another non-limiting embodiment, a mixture of methanol and dimethyl ether is reacted with carbon monoxide in a first reaction zone to provide a reaction product comprising methyl acetate, acetic acid, and unreacted methanol. The reaction product then is passed to a second reaction zone, whereby the acetic acid is reacted with the unreacted methanol to provide methyl acetate.

In such a non-limiting embodiment, although the scope of the present invention is not intended to be limited to any theoretical reasoning, it is believed that, while each of the methanol and the dimethyl ether is being reacted with the carbon monoxide in the first reaction zone to produce methyl acetate, a portion of the methanol in the first reaction zone is dehydrated to form dimethyl ether according to the following equation:

2CH₃OH→CH₃OCH₃+H₂O

The dimethyl ether, including both “fresh” dimethyl ether and dimethyl ether formed as a result of the dehydration of a portion of the methanol, is reacted with carbon monoxide to form methyl acetate according to the following equation:

CH₃OCH₃+CO→CH₃COOCH₃

In a non-limiting embodiment, the reaction product further comprises unreacted dimethyl ether. In another non-limiting embodiment, the reaction product further comprises water. In yet another non-limiting embodiment, the water is removed prior to reacting the acetic acid with the unreacted methanol to provide additional methyl acetate.

In another non-limiting embodiment, the reaction product further comprises unreacted carbon monoxide.

In yet another non-limiting embodiment, additional “fresh” methanol is fed to the second reaction zone without having been passed through the first reaction zone.

In another non-limiting embodiment, dimethyl ether is reacted with carbon monoxide and water in a first reaction zone to provide a reaction product comprising methyl acetate, acetic acid, methanol, and unreacted dimethyl ether.

The reaction product then is passed to the second reaction zone. In the second reaction zone, the acetic acid in the reaction product is reacted with methanol to provide methyl acetate.

In a non-limiting embodiment, the amount of water does not exceed 3 wt. % of the total amount of dimethyl ether, carbon monoxide, and water that is passed to the first reaction zone.

In such a non-limiting embodiment, although the scope of the present invention is not to be limited to any theoretical reasoning, a portion of the dimethyl ether is reacted with carbon monoxide in the first reaction zone to produce methyl acetate as hereinabove described. Another portion of the dimethyl ether is reacted in the first reaction zone with water to form methanol according to the following equation:

CH₃OCH₃+H₂O→2CH₃OH.

The methanol then is reacted with carbon monoxide to form methyl acetate and acetic acid as hereinabove described. Any unreacted methanol then can be reacted in the second reaction zone to produce additional methyl acetate.

In another non-limiting embodiment, additional, or “fresh”, methanol is passed to the second reaction zone without having been passed through the first reaction zone. This “fresh” methanol, along with any methanol from the first reaction zone, is reacted with acetic acid to produce additional methyl acetate.

In a non-limiting embodiment, any unreacted water that is remaining after the reactions that occurred in the first reaction zone is removed from the reaction product prior to reacting the acetic acid with methanol to provide methyl acetate.

In another non-limiting embodiment, the reaction product further comprises unreacted carbon monoxide.

In a non-limiting embodiment, the first reaction zone and the second reaction zone are contained in a single reaction vessel. In another non-limiting embodiment, the first reaction zone is contained in a first reaction vessel, and the second reaction zone is contained in a second reaction vessel.

In another non-limiting embodiment, the carbon monoxide is obtained from synthesis gas. In a non-limiting embodiment, the synthesis gas is produced by gasifying a biomass-rich material. Representative examples of producing synthesis gas by gasifying a biomass-rich material are disclosed in published PCT Application Nos. WO2009/132449 and WO2010/069068, the contents of which are incorporated herein by reference.

In a non-limiting embodiment, the at least one reactant containing at least one methyl group is reacted with carbon monoxide in the first reaction zone at a molar ratio of the at least one reactant to carbon monoxide of from about 0.5 to about 10. In another non-limiting embodiment, the at least one reactant is reacted with carbon monoxide at a molar ratio of the at least one reactant to carbon monoxide of from about 0.7 to about 4.

In a non-limiting embodiment, the at least one reactant containing at least one methyl group and carbon monoxide are reacted in the first reaction zone at a temperature of from about 100° C. to about 500° C. In another non-limiting embodiment, the at least one reactant and carbon monoxide are reacted at a temperature of from about 200° C. to about 300° C.

In a non-limiting embodiment, the at least one reactant containing at least one methyl group and carbon monoxide are reacted at a pressure of from about 1 atm to about 100 atm. In another non-limiting embodiment, the at least one reactant and carbon monoxide are reacted at a pressure of from about 5 atm to about 50 atm.

In a non-limiting embodiment, the at least one reactant containing at least one methyl group and carbon monoxide are reacted at a gas hourly space velocity (GHSV), of from about 0.1 h⁻¹ to about 10,000 h⁻¹. In another non-limiting embodiment, the at least one reactant and carbon monoxide are reacted at a GHSV of from about 0.7 h⁻¹ to about 6,000 h⁻¹.

In another non-limiting embodiment, the at least one reactant containing at least one methyl group and carbon monoxide are reacted at a gas hourly space velocity (GHSV), based on the gas (mainly carbon monoxide) at a gas hourly space velocity of from about 10 h⁻¹ to about 2,000 h⁻¹. In a further non-limiting embodiment, the at least one reactant and carbon monoxide are reacted at a GHSV of from about 250 h⁻¹ to about 1,500 h⁻¹. In yet another non-limiting embodiment, the at least one reactant and carbon monoxide are reacted at a GHSV of from about 350 h⁻¹ to about 1,000 h⁻¹.

As noted hereinabove, the carbon monoxide may be obtained from synthesis gas. Thus, in a non-limiting embodiment, the reaction of the at least one reactant including at least one methyl group with carbon monoxide in the first reaction zone is effected by reacting the at least one reactant with a syngas, such as CO-rich syngas.

The at least one reactant including at least one methyl group and carbon monoxide are reacted in the first reaction zone in the presence of a suitable catalyst for converting the at least one reactant and carbon monoxide to methyl acetate and acetic acid.

In a non-limiting embodiment, the catalyst may be a salt of an active metal, or a finely divided and/or slurried powdered active metal. Such active metals include, but are not limited to, Group VIII metals such as Co, Ni, Pd, Ru, Rh, Re, Os, Ir, and the like. In a non-limiting embodiment, the active metal may be supported on an appropriate support including, but not limited to, carbon, alumina, silica, chromite, zirconia, or other stable oxides such as iron oxide, molybdenum oxide, and the like. The active metal, in a non-limiting embodiment, may be employed in combination with a promoter, such as a halide (e.g., bromide, chloride, iodide). In one non-limiting embodiment, the halide is an organic halide such as a methyl halide. In another non-limiting embodiment, the halide is a metal halide. In another non-limiting embodiment, the promoter is a “green promoter”, such as, for example, a dimethyl carbonate promoter. In another non-limiting embodiment, the active metal may be employed in combination with other additives, such as alkali metals (e.g. Li, Na, K, Rh, Cs), alkaline earth metals (eg, Ba, Mg, Cs, Sr, Ra), and/or promoter metals such as Mo, Cu, Au, Ag, W, V, Cd, Cr, Zn, Mn, or Sn.

In another non-limiting embodiment, the catalyst is suspended or dispersed in an inert liquid medium, such as, for example, an inert oil.

In a non-limiting embodiment, the catalyst is rhodium supported on carbon (which may be activated carbon) or alumina. The rhodium is impregnated on the support such that there is provided from about 0.1 wt. % to about 2 wt. % of rhodium on the support. The support also is impregnated with an alkali or alkali iodide at a molar ratio of from about 0.1 to about 5 with respect to the rhodium. The catalyst also may be employed in combination with a halide promoter as hereinabove described.

The reaction of the at least one reactant including at least one methyl group and carbon monoxide provides a reaction product which includes methyl acetate, acetic acid, and unreacted methanol and/or unreacted dimethyl ether. As noted hereinabove, the reaction product also may include water, which may be in the form of water vapor. In another non-limiting embodiment, the reaction product also may include unreacted carbon monoxide.

In a non-limiting embodiment, the water, if present, is removed from the reaction product prior to reacting the acetic acid with methanol. In one non-limiting embodiment, the water is removed from the reaction product by contacting the reaction product with a desiccant. Suitable desiccants which may be employed include, but are not limited to, zeolites, such as, for example, Linde Type A (LTA) zeolite, zeolite 3A, zeolite 4A, zeolite 5A, zeolite 13X, or chabazite, activated alumina, activated carbon, silica gel, oxides such as calcium oxide or magnesium oxide, sodium sulfate, magnesium sulfate, calcium sulfate, magnesium carbonate, or calcium carbonate. In a non-limiting embodiment, the dessicant is selected from the group consisting of LTA zeolite, zeolite 3A, and zeolite 4A.

In a non-limiting embodiment, the reaction product is contacted with a bed of the desiccant in a pressure swing adsorption zone, wherein the reaction product is passed over the bed of the desiccant at a pressure of from about 1 atm to about 100 atm. At such pressure, water is adsorbed into the desiccant material. In a non-limiting embodiment, the pressure is from about 2 atm to about 8 atm. Once the desiccant bed has reached its adsorptive capacity, the passage of the reaction product over the desiccant bed is stopped, and the desiccant bed is regenerated by decreasing the pressure of the pressure swing adsorption zone to a pressure which is lower than the pressure at which the water was adsorbed, whereby water is released or desorbed from the desiccant bed. In a non-limiting embodiment, such pressure is from about 1 atm to about 7 atm.

In another non-limiting embodiment, the reaction product is contacted with a bed of the desiccant in a temperature swing adsorption zone wherein the reaction product is passed over the bed of the desiccant at a temperature of from about 90° C. to about 150° C., whereby water is adsorbed into the desiccant bed. Once the desiccant bed has reached its adsorptive capacity, the passage of reaction product over the desiccant bed is stopped, and the desiccant bed is regenerated by raising the temperature of the temperature swing adsorption zone to a temperature which is higher than the temperature at which the water was adsorbed, whereby water is released or desorbed from the desiccant bed. In a non-limiting embodiment, such temperature is from about 100° C. to about 200° C.

In another non-limiting embodiment, when a pressure swing or temperature swing adsorption zone is employed, there are provided two or more parallel desiccant beds. The reaction product is passed through one of the desiccant beds, whereby water is removed from the reaction product, while the other desiccant bed(s), in parallel, is (are) being regenerated under pressure swing or temperature swing conditions hereinabove described. Thus, when a desiccant bed has reached its adsorptive capacity, flow of the reaction product is shifted to the other desiccant bed(s), such as, for example, via a valve system, while the bed which has reached its adsorptive capacity is regenerated. Thus, in such an embodiment, the process does not need to be stopped while the desiccant bed is regenerated.

In a non-limiting embodiment, the acetic acid and methanol are reacted in the second reaction zone at a temperature of from about 100° C. to about 500° C. In another non-limiting embodiment, the acetic acid and methanol are reacted in the second reaction zone at a temperature of from about 120° C. to about 300° C.

In a non-limiting embodiment, the acetic acid is reacted with the methanol in the second reaction zone at a pressure of from about 1 atm to about 100 atm. In another non-limiting embodiment, the acetic acid is reacted with methanol in the second reaction zone at a pressure of from about 1 atm to about 50 atm.

In another non-limiting embodiment, the acetic acid is reacted with the methanol in the second reaction zone at a gas hourly space velocity (GHSV) of from about 0.1 h⁻¹ to about 10,000 h⁻¹. In another non-limiting embodiment, the acetic acid is reacted with the unreacted methanol in the second reaction zone at a GHSV of from about 1 h⁻¹ to about 6,000 h⁻¹.

The acetic acid and methanol are reacted in the second reaction zone in the presence of a suitable catalyst. Catalysts which may be employed in the second reaction zone include, but are not limited to, zeolite catalysts and other porous or mesoporous materials related to aluminosilicate, as well as rhodium, which may be supported on an appropriate support, such as activated carbon. In a non-limiting embodiment, the zeolite catalyst is selected from the group consisting of faujasite zeolites, zeolite Beta, Linde Type L (LTL) zeolite, and MCM-41. In another non-limiting embodiment, the zeolite is in protonic form. In yet another non-limiting embodiment, the zeolite is exchanged partially with a cation, such as, for example, sodium, lithium, potassium, or cesium.

In accordance with another aspect of the present invention, there is provided a method of producing at least one acetate from at least one alcohol and/or at least one ether. The method comprises reacting the at least one alcohol and/or at least one ether with carbon monoxide in a first reaction zone to provide a reaction product comprising at least one acetate, at least one acid, and unreacted at least one alcohol and/or unreacted at least one ether. The reaction product then is passed to a second reaction zone, whereby the at least one acid is reacted with at least one alcohol to provide at least one acetate.

In a non-limiting embodiment, the at least one alcohol in the first reaction zone is methanol, the at least one acid is acetic acid, and the at least one acetate is methyl acetate. In another non-limiting embodiment, the at least one alcohol in the first reaction zone is ethanol, the at least one acid is propionic acid, and the at least one acetate is ethyl acetate.

In a non-limiting embodiment, the at least one alcohol is reacted with carbon monoxide in the first reaction zone to provide a reaction product which comprises at least one acetate, at least one acid, unreacted at least one alcohol, and water.

In another non-limiting embodiment, the water is removed prior to reacting the at least one acid with the unreacted at least one alcohol in the second reaction zone.

In other non-limiting embodiment, the reaction product further comprises unreacted carbon monoxide.

In a non-limiting embodiment, the at least one alcohol that is reacted in the second reaction zone includes unreacted alcohol from the first reaction zone, such as unreacted methanol or unreacted ethanol.

In another non-limiting embodiment, the at least one alcohol that is reacted in the second reaction zone includes “fresh” alcohol that is fed directly to the second reaction zone, such as “fresh” methanol or “fresh” ethanol.

In yet another non-limiting embodiment, methanol is reacted with carbon monoxide in the first reaction zone to produce a product which includes methyl acetate, acetic acid, and water, as well as unreacted methanol. The reactions of methanol with carbon monoxide in the first reaction zone are as follows:

2CH₃CH+CO→CH₃COOCH₃+H₂O

CH₃OH+CO→CH₃COOH

The acetic acid then is reacted with fresh ethanol in the second reaction zone to produce ethyl acetate. Such reaction is as follows:

CH₃COOH+CH₃CH₂OH→CH₃CH₂COOCH₃+H₂O

In a non-limiting embodiment, the first reaction zone and the second reaction zone are contained in a single reaction vessel. In another non-limiting embodiment, the first reaction zone is contained in a first reaction vessel, and the second reaction zone is contained in a second reaction vessel.

In a non-limiting embodiment, the at least one alcohol and/or at least one ether is reacted with carbon monoxide in the first reaction zone under the same temperature, pressure, and space velocity conditions hereinabove described with respect to reacting at least one reactant including at least one methyl group with carbon monoxide in the first reaction zone to provide a reaction product including methyl acetate and acetic acid. The first reaction zone, in a non-limiting embodiment, may include the same catalysts hereinabove described, which were employed for catalyzing the reaction of at least one reactant including at least one methyl group with carbon monoxide to provide methyl acetate and acetic acid.

In another non-limiting embodiment, the at least one acid is reacted with the at least one alcohol in the second reaction zone under the same temperature, pressure, and space velocity conditions hereinabove described with respect to reacting acetic acid with methanol in the second reaction zone to provide methyl acetate. The second reaction zone, in a non-limiting embodiment, may include the same catalysts as hereinabove described, which were employed for catalyzing the reaction of acetic acid with methanol to provide methyl acetate.

The invention now will be described with respect to the drawings, wherein:

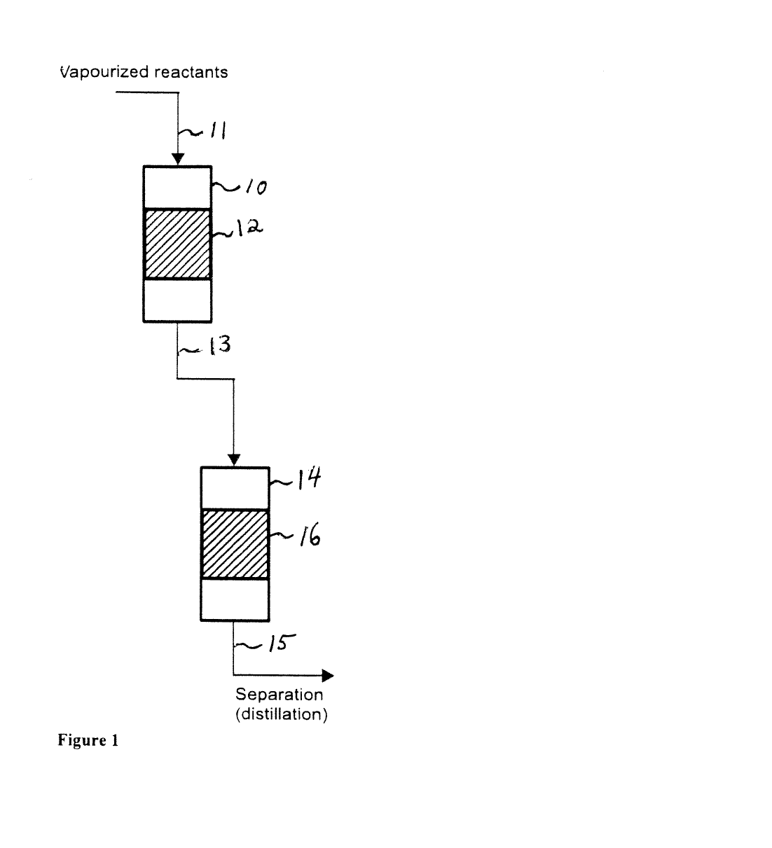

FIG. 1 is a schematic of a first embodiment of the method of the present invention;

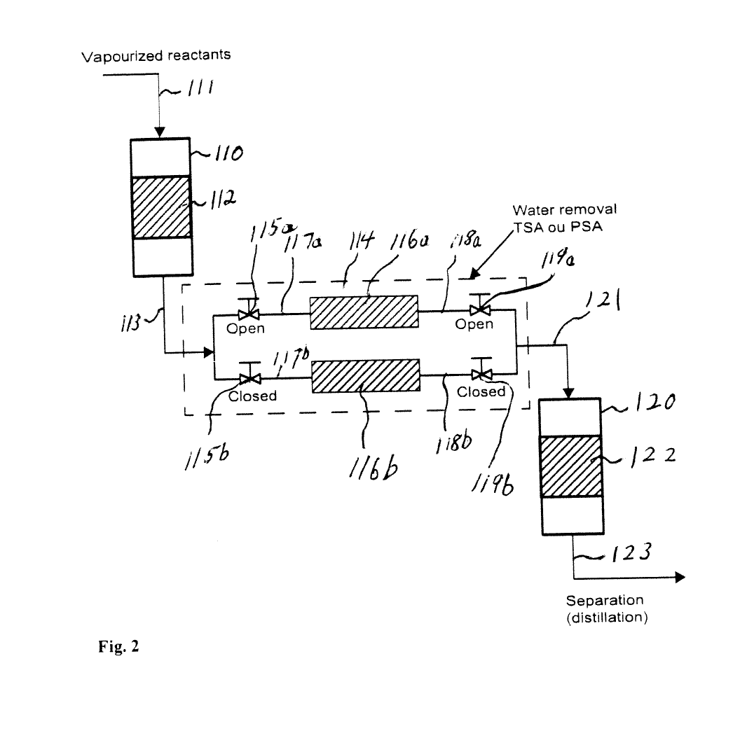

FIG. 2 is a schematic of a second embodiment of the method of the present invention; and

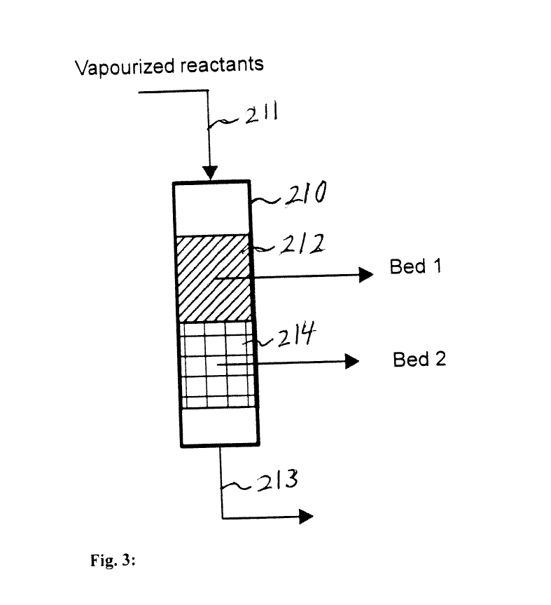

FIG. 3 is a schematic of a third embodiment of the method of the present invention.

Referring now to the drawings, vaporized methanol and carbon monoxide in line 11 are fed to reactor 10, which includes a catalyst bed 12 of an appropriate catalyst as hereinabove described. The methanol and carbon monoxide are reacted in the presence of the catalyst contained in catalyst bed 12 under conditions to provide a reaction product that includes methyl acetate, acetic acid, unreacted methanol, and water. The reaction product is withdrawn from reactor 10 through line 13 and passed to esterification reactor 14, which contains a catalyst bed 16, containing an appropriate esterification catalyst as hereinabove described. The acetic acid and unreacted methanol are reacted in the presence of the catalyst contained in catalyst bed 16 under conditions to provide additional methyl acetate. The resulting product is withdrawn from esterification reactor 14 through line 15. The product then may be subjected to further processing, such as distillation, for example, in order to provide a purified methyl acetate product.

In another non-limiting embodiment, as shown in FIG. 2, vaporized methanol and carbon monoxide in line 111 are passed to reactor 110, which contains a catalyst bed 112. The methanol and carbon monoxide are reacted in the presence of the catalyst contained in catalyst bed 112 under conditions hereinabove described to produce a reaction product including methyl acetate, acetic acid, unreacted methanol, and water. This reaction product is withdrawn from reactor 110 through line 113, and passed to a water removal zone, indicated schematically at 114.

Water removal zone 114 includes desiccators 116a and 116b, each of which contain a desiccant as hereinabove described. The desiccators 116a and 116b can be operated by means of pressure swing adsorption or temperature swing adsorption under the pressure or temperature conditions hereinabove described. As shown in FIG. 2, the reaction product in line 113 is passed through line 117a and open valve 115a to desiccator 116a, whereby water is adsorbed by the desiccant contained therein. At the same time, valves 115b and 119b are closed, thereby preventing the passage of reaction product through line 117b, desiccator 116b, and line 118b, while the desiccant in 116b is being regenerated, either through a decrease in pressure or an increase in temperature in order to desorb water from the desiccant in desiccator 116b.

Reaction product is passed through desiccator 116a until the desiccant contained therein has reached its adsorptive capacity, at which time valves 115a and 119a are closed, and valves 115b and 119b are opened, thereby permitting the passage of reaction product over the regenerated desiccant in desiccator 116b.

After the reaction product is passed through desiccator 116a to remove water therefrom, the reaction product, which includes methyl acetate, acetic acid, and unreacted methanol, is passed through line 118b, open valve 119b, and line 121 to esterification reactor 120, which contains a catalyst bed 122 of an appropriate esterification catalyst as hereinabove described. The acetic acid and unreacted methanol are reacted in the presence of the catalyst contained in catalyst bed 122 in esterification reactor 120 to provide additional methyl acetate. The resulting product is withdrawn from esterification reactor 120 through line 123. The product then may be subjected to further processing, such as distillation, for example, in order to provide a purified methyl acetate product.

In another non-limiting embodiment, as shown in FIG. 3, vaporized methanol and carbon monoxide in line 211 are passed to reactor 210. Reactor 210 includes a first catalyst bed 212, which contains an appropriate catalyst for converting methanol and carbon monoxide to methyl acetate and acetic acid, and a second catalyst bed 214 which contains an appropriate esterification catalyst, for catalyzing the esterification reaction of acetic acid with unreacted methanol to provide additional methyl acetate.

Thus, the methanol and carbon monoxide are reacted in the presence of the catalyst in first catalyst bed 212 to provide a reaction product including methyl acetate, acetic acid, unreacted methanol, and water. This reaction product then is passed from the first catalyst bed 212 to the second catalyst bed 214, whereby the acetic acid is reacted with the unreacted methanol to provide additional methyl acetate. The resulting product then is withdrawn from reactor 210 through line 213, and then may be subjected to further processing, such as distillation, for example, to provide a purified methyl acetate product.

The invention now will be described with respect to the following examples; however, the scope of the present invention is not intended to be limited thereby.

### EXAMPLE 1

### Carbonylation of Methanol

A feed of 67.89 mole % methanol, 22.63 mole % carbon monoxide, 5.15 mole % nitrogen, 2.22 mole % hydrogen, and 2.11 mole % of a methyl iodide promoter was fed to a carbonylation reactor that was operated at a temperature of 215° C. and a pressure of 27.2 atm. The feed was passed through the reactor at a gas hourly space velocity (GHSV), based on CO, of 500 l/hr. The feed was reacted for 6 hours in a presence of a fixed bed of a rhodium catalyst supported on activated carbon. The volume of catalyst in the fixed bed was 120 ml.

The above reaction produced a product of 38.32 mole % methanol, 25.12 mole % methyl acetate, 32.58 mole % water, 1.67 mole % acetic acid, and 2.29 mole % methyl iodide. Overall methanol conversion was 57.11% and overall CO conversion was 89.98%. The conversion of CO to methyl acetate was 84.37%, and the conversion of CO to acetic acid was 5.61%. The methyl acetate selectivity was 93.8%.

### EXAMPLE 2

### Esterification of Acetic Acid

A feed of 44.28 mole % methanol, 29.08 mole % methyl acetate, 22.14 mole % carbon monoxide, 2.58 mole % methyl iodide, and 1.91 mole % acetic acid was fed to an esterification reactor that was operated at a temperature of 140° C. and a pressure of 80 psi. The feed was passed through the esterification reactor at a gas hourly space velocity, based on CO, of 400 l/hr. The feed was reacted for 4 hours in the presence of a fixed bed of rhodium catalyst supported on activated carbon. The volume of catalyst in the fixed bed was 120 ml.

The above reaction produced a product of 49.28 mole % methyl acetate, 41.15 mole % methanol, 6.14 mole % water, and 3.43 mole % methyl iodide.

### EXAMPLE 3

The carbonylation of methanol with carbon monoxide is carried out in a fixed bed reactor in which two types of catalysts are tested: rhodium on carbon (which may be activated carbon) and rhodium on alumina. The carbon or alumina support is impregnated with the rhodium in order to provide 0.5 wt. % to 1 wt. % of rhodium on the support. The support also is impregnated with an alkali or alkali iodide at a molar ratio of 2 to 5 with respect to the rhodium impregnated previously on the support. The catalyst is calcined at 350° C. under a hydrogen or nitrogen atmosphere for about 4 to about 20 hours. The catalyst then is placed between two zones filled with deionized carborundum or another inert material.

The carbonylation of methanol with carbon monoxide is carried out in the presence of a methyl iodide promoter, which is added to the methanol feed in a molar ratio of methyl iodide to methanol of from about 0.05:1 to about 5:1. The carbonylation also is carried out at a temperature of from 200° C. to 300° C., pressure of from 5 atm to 50 atm, a methanol to CO molar ratio of from 0.7 to 4, and a GHSV (based on CO) of from 350 to 1,000 h⁻¹. The reaction product includes the following components in the following amounts:

methanol: 1-35 mol %

methyl iodide: 1.5-2.5 mol %

methyl acetate: 3-30 mol %

acetic acid: 1-77 mol %

water: 16-32 mol %

The unreacted methanol and the acetic acid in the reaction product then are reacted in the presence of a zeolite catalyst, such as a faujasite zeolite, zeolite Beta, or Linde Type L (LTL) zeolite, in protonic form, or MCM-41. The unreacted methanol and acetic acid are reacted in the presence of the zeolite catalyst at a temperature of from 120° C. to 300° C., a pressure of from 1 atm to 50 atm, and at a GHSV, based on the methanol, of from 1 h⁻¹ to 6,000 h⁻¹, to provide additional methyl acetate.

The disclosures of all patents and publications, including published patent applications, are incorporated herein by reference to the same extent as if each patent and publication were incorporated individually by reference.

It is to be understood, however, that the scope of the present invention is not to be limited to the specific embodiments described above. The invention may be practiced other than as described particularly and still be within the scope of the accompanying claims.

1. A method of producing methyl acetate from methanol, comprising:
(a) reacting methanol with carbon monoxide in a first reaction zone to provide a reaction product comprising methyl acetate, acetic acid, and unreacted methanol; and
(b) passing said reaction product from step (a) to a second reaction zone, whereby said acetic acid is reacted with said unreacted methanol from step (a) to provide methyl acetate, wherein said first reaction zone and said second reaction zone are contained in a single reaction vessel.

2. The method of claim 1 wherein said reaction product of step (a) further comprises water.

3. The method of claim 1 wherein said reaction product of step (a) further comprises unreacted carbon monoxide.

4. The method of claim 2 wherein prior to step (b), said water is removed from said reaction product of step (a).

5.-6. (canceled)

7. The method of claim 1 wherein said carbon monoxide is obtained from synthesis gas.

8. A method of producing methyl acetate from methanol, comprising:
(a) reacting a mixture of methanol and dimethyl ether with carbon monoxide in a first reaction zone to provide a reaction product comprising methyl acetate, acetic acid, and unreacted methanol; and
(b) passing said reaction product from step (a) to a second reaction zone, whereby said acetic acid is reacted with said unreacted methanol from step (a) to provide methyl acetate, wherein said first reaction zone and said second reaction zone are contained in a single reaction vessel.

9. The method of claim 8 wherein said reaction product of step (a) further comprises water.

10. The method of claim 9 whereas, prior to step (b), said water is removed from said reaction product of step (a).

11. The method of claim 8 wherein said reaction product of step (a) further comprises unreacted dimethyl ether.

12. The method of claim 8 where said reaction product of step (a) further comprises unreacted carbon monoxide.

13. The method of claim 8 wherein, in step (b), additional methanol has been passed to said second reaction zone without having been passed through said first reaction zone.

14.-15. (canceled)

16. The method of claim 8 wherein said carbon monoxide is obtained from synthesis gas.

17. A method of producing at least one acetate from at least one alcohol and/or at least one ether, comprising:
(a) reacting said at least one alcohol and/or at least one ether with carbon monoxide in a first reaction zone to provide a reaction product comprising at least one acetate, at least one acid, and unreacted at least one alcohol and/or unreacted at least one ether; and
(b) passing said reaction product from step (a) to a second reaction zone, whereby said at least one acid is reacted with at least one alcohol to provide at least one acetate, wherein said first reaction zone and said second reaction zone are contained in a single reaction vessel.
